(19) United States
(12) Patent Application Publication
(10) Pub. No.: US 2009/0099154 A1
(43) Pub. Date: Apr. 16, 2009

(54) Pharmaceutical Sustained Release Compositions and Processes Thereof

(75) Inventors: Rajesh Jain, New Delhi (IN); Kour Chand Jindal, New Delhi (IN); Sukhjeet Singh, New Delhi (IN); Sanjay Boldhane, New Delhi (IN)

Correspondence Address: LADAS & PARRY LLP, 26 WEST 61ST STREET, NEW YORK, NY, 10023, US

(73) Assignee: PANACEA BIOTEC LTD., New Delhi (IN)

(21) Appl. No.: 11/922,960

(22) PCT Filed: Jun. 29, 2006
(86) PCT No.: PCT/IN2006/000225

§ 371 Date: Dec. 27, 2007

(30) Foreign Application Priority Data
Jun. 29, 2005 (IN) 1680/DEL/2005

(51) Int. Cl. A61K 31/52; A61K 31/522; A61K 31/341; A61K 31/445; A61K 31/40; A61K 31/135; A61K 31/55; A61K 31/4015; A61P 31/12

(52) U.S. Cl. 514/217; 514/263.4; 514/263.38; 514/471; 514/317; 514/423; 514/649; 514/424

(57) ABSTRACT

Pharmaceutical sustained release composition comprising at least one active agent(s), or its tautomeric forms, analogues, isomers, polymorphs, solvates, or salts thereof; preferably an antiviral active agent is provided. Also provided is a process of preparation of such composition and method of using them. The sustained release compositions of the present invention are able to deliver the active agent in a desired manner for an extended period of time.

### FIELD OF THE INVENTION

The present invention relates to novel pharmaceutical sustained release compositions and process of preparation of such compositions preferably comprising active agent(s) having good bioavailability. Particularly this invention pertains to pharmaceutical compositions comprising antiviral active agent, process of preparation of such compositions and method of using them.

### BACKGROUND OF THE INVENTION

The advantages of sustained release products are well known in the pharmaceutical field and include the ability to slowly release the medicament over a period of time while increasing patient compliance by reducing the number of administrations necessary to achieve the same level. Various attempts to provide dosage forms for delivery of active agent that remain in the stomach for extended periods or time, have been described previously.

U.S. Pat. No. 4,851,232 describes a hydrogel reservoir containing tiny pills having an active agent core surrounded by a wall controlling delivery of active agent to the stomach. The hydrogel swells in the stomach to facilitate retention of the active agent reservoir in the stomach over time. U.S. Pat. No. 4,871,548 describes a dosage form including a mixture of low and high number average molecular weight hydroxypropyl methylcellulose polymers and active agent that swells when in the stomach. U.S. Pat. No. 6,548,083 describes a gastro-retentive controlled release dosage form comprising an active agent and a polymer matrix formed of a mixture of a swellable, water soluble polymer such as polyethylene oxide and cellulosic polymer derivatives including hydroxypropyl cellulose, hydroxypropyl methylcellulose, hydroxyethyl cellulose, sodium carboxymethylcellulose, calcium carboxymethylcellulose, methyl cellulose, as well as noncellulosics such as maltodextrin, polyvinyl alcohol, polyacrylic acids, alginates, gelatin, natural gums, that expands when in contact with fluids in the gastric environment and a hydro attractant such as low substituted hydroxypropyl cellulose, ion exchange resins, microcrystalline cellulose, etc. U.S. Pat. Nos. 6,395,303 and 6,866,867 describe improved process for the preparation of an agglomerated solid dosage form to deliver active ingredients such as locally active agents like antifungals, antibiotics and antiviral agents. US Publication. No. 2003215496 describes a pharmaceutical composition in the form of a solid carrier comprising a substrate and an encapsulation coat on the substrate comprising a therapeutically effective amount of a hydrophobic pharmaceutical active ingredient and an effective solubilizing amount of at least one hydrophilic surfactant, which is an amount effective to facilitate sustained solubilization of the active ingredient upon administration. US Publication. No. 2004185105 describes a method for selecting an optimized controlled release dosage form for administration to a patient having a predetermined drug release profile in vivo by preparing a plurality of different candidate dosage forms each comprised of a biocompatible, hydrophilic polymer and a pharmacologically active agent incorporated therein. U.S. Pat. No. 5,007,790 describes a sustained-release oral drug dosage form for releasing a solution of drug into the stomach comprising a plurality of solid particles of a solid-state drug dispersed within a hydrophilic, water-swellable polymer.

Several antiviral active agents exist such as famciclovir, valacyclovir, penciclovir, ganciclovir, and the like. Famciclovir is an oral and the diacetyl 6-deoxy prodrug of the antiherpesvirus nucleoside analogue, penciclovir which is active against the Herpes viruses, including herpes simplex 1 and 2 (cold sores and genital herpes) and varicella-zoster (shingles and chicken pox). It is the penciclovir that is active against the viruses. Penciclovir is phosphorylated by viral thymidine kinase to penciclovir monophosphate, which is then converted to penciclovir triphosphate by cellular kinases. It inhibits the replication of viral DNA that is necessary in order for viruses to reproduce themselves. Famciclovir is active against the same viruses as acyclovir but has a longer duration of action. Therefore, it can be taken fewer times each day. Famciclovir was approved for use by the USFDA in 1994. Famciclovir undergoes rapid biotransformation to the active antiviral compound penciclovir, which has inhibitory activity against herpes simplex virus types I (HSV-1) and 2 (HSV-2) and varicella zoster virus (VZV). Penciclovir triphosphate has an intracellular half-life of 10 hours in HSV-1-, 20 hours in HSV-2- and 7 hours in VZV-infected cells cultured in vitro; however, the clinical significance is unknown. Acyclovir is a synthetic purine nucleoside analogue with in vitro and in vivo inhibitory activity against herpes simplex virus types I (HSV-1), 2 (HSV-2), and varicella-zoster virus (VZV). Valacyclovir hydrochloride is the hydrochloride salt of L-valyl ester of the antiviral drug acyclovir. Valacyclovir is used to treat cold sores (herpes labialis) and shingles (herpes zoster). It is also used to treat genital herpes in patients with a normal immune system. Cimetidine is a histamine H₂-receptor antagonist that competitively inhibits the action of histamine at the histamine H₂ receptors of the parietal cells. Metformin is an antihyperglycemic agent, which improves glucose tolerance in patients with type 2 diabetes, lowering both basal and postprandial plasma glucose. Metformin decreases hepatic glucose production, decreases intestinal absorption of glucose, and improves insulin sensitivity by increasing peripheral glucose uptake and utilization. Captopril is a specific competitive inhibitor of angiotensin I-converting enzyme (ACE), the enzyme responsible for the conversion of angiotensin I to angiotensin II. Its beneficial effects in hypertension and heart failure are primarily from suppression of the renin-angiotensin-aldosterone system. Captopril prevents the conversion of angiotensin I to angiotensin II by inhibition of ACE. Bupropion is an antidepressant of the aminoketone class, chemically unrelated to tricyclics or selective serotonin reuptake inhibitors. Bupropion is both a dopamine reuptake inhibitor and a norepinephrine reuptake inhibitor, and is often used as a smoking cessation aid. Tramadol is a centrally acting synthetic opioid analgesic and works by two complementary mechanisms which include binding of parent and M1 metabolite to μ-opioid receptors and weak inhibition of reuptake of norepinephrine and serotonin. Oxcarbazepine is an antiepileptic drug, which primarily exerts its actions through its 10-monohydroxy metabolite (MHD). Oxcarbazepine and its metabolite MHD exert their antiseizure effect by blockade of voltage-sensitive sodium channels, resulting in stabilization of hyperexcited neural membranes, inhibition of repetitive neuronal firing, and diminution of propagation of synaptic impulses. Levetiracetam is an antiepileptic drug indicated as adjunctive therapy in the treatment of partial onset seizures in adults and children 4 years of age and older with epilepsy. Fexofenadine hydrochloride is an antihistaminic drug used in treatment of hayfever and allergy symptoms.

Although several prior art literature is available on oral sustained release delivery systems; there still exists a need for developing sustained release systems for delivery of drugs, which releases the drug in a specific desired manner consistently and uniformly. The inventors of the present invention with considerable expense of intellectual effort have done extensive research and conducted several experiments using sustaining systems comprising of different polymers alongwith other suitable excipients in which the antiviral agent is formulated thus providing sustained release systems that have a significant advancement over the prior art.

### SUMMARY OF THE INVENTION

It is an objective of the present invention to provide novel pharmaceutical sustained release composition comprising at least one active agent(s) or its tautomeric forms, analogues, isomers, polymorphs, solvates, derivatives, or salts thereof; at least one pH independent polymer(s); a sustaining system comprising at least a gum; and optionally one or more pharmaceutically acceptable excipients.

It is an objective of the present invention to provide novel pharmaceutical sustained release composition comprising at least one active agent(s) or its tautomeric forms, analogues, isomers, polymorphs, solvates, derivatives, or salts thereof; at least one pH independent polymer(s); a sustaining system comprising at least a gum and a methacrylic acid polymer; and optionally one or more pharmaceutically acceptable excipients.

It is an objective of the present invention to provide novel pharmaceutical sustained release composition comprising at least one active agent(s) or its tautomeric forms, analogues, isomers, polymorphs, solvates, derivatives, or salts thereof; at least one pH independent polymer(s); a sustaining system comprising at least a gum; at least one filler(s); at least one inorganic salt(s); and optionally one or more pharmaceutically acceptable excipients.

It is also an objective of the present invention to provide novel pharmaceutical sustained release composition comprising at least one active agent(s) preferably selected from a group comprising antivirals, antiulcers, antihypertensives, antidiabetics, antidepressants, antihistaminics, antiepileptics, analgesics, or its tautomeric forms, analogues, isomers, polymorphs, solvates, derivatives, or salts thereof; at least one pH independent polymer(s); a sustaining system comprising at least a gum; and optionally one or more pharmaceutically acceptable excipients.

It is also an objective of the present invention to provide novel pharmaceutical sustained release composition comprising at least one active agent(s) preferably an antiviral agent selected from a group comprising acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; leveteracetam; tramadol; and the like or their tautomeric forms, analogues, isomers, polymorphs, solvates, derivatives, or salts thereof.

It is also an objective of the present invention to provide novel pharmaceutical sustained release composition comprising famciclovir or its tautomeric forms, analogues, isomers, polymorphs, solvates, derivatives, or salts thereof as the active agent; at least one pH independent polymer(s); a sustaining system comprising at least a gum; and optionally one or more pharmaceutically acceptable excipients.

It is another objective of the present invention to provide process for preparation of such compositions.

It is a further objective of the present invention to provide process for preparation of such composition, which comprises of the following steps:
i. Granulation of active agent(s) or optionally a mixture of active agent(s) with a pH independent polymer(s),
ii. Mixing the granules thus obtained with sustaining system, optionally with inorganic salt(s), and/or other pharmaceutically acceptable excipients, and
iii. Formulation of the mixture into a suitable dosage form.

It is yet another objective of the present invention to provide method of using such compositions which comprises administering to a patient in need thereof an effective amount of the composition.

### DETAILED DESCRIPTION OF THE INVENTION

The present invention provides novel pharmaceutical sustained release composition comprising at least one active agent. In an embodiment, the active agent(s) is selected from but not limited to a group comprising antivirals, antiulcers, antihypertensives, antidiabetics, CNS depressants, antihistaminics, anticonvulsants, analgesics or its tautomeric forms, analogues, isomers, polymorphs, solvates, or salts thereof.

In an embodiment of the present invention is provided a novel pharmaceutical sustained release composition comprising at least one active agent(s) preferably an antiviral agent selected from a group comprising acyclovir, famciclovir, valacyclovir, penciclovir, ganciclovir, ritonavir, lopinavir, saquinavir, and the like; cimetidine; ranitidine; captopril; metformin; bupropion; fexofenadine; oxcarbazepine; leveteracetam; tramadol; and the like or their tautomeric forms, analogues, isomers, polymorphs, solvates, derivatives, or salts thereof. Preferably the active agent is an antiviral agent, more preferably famciclovir.

The compositions of the present invention comprises of an active agent(s) or its tautomeric forms, analogues, isomers, polymorphs, solvates, derivatives, or salts thereof; at least one pH independent polymer(s); a sustaining system comprising at least a gum; and optionally one or more pharmaceutically acceptable excipients. In an embodiment, the sustaining system additionally comprises a methacrylic acid polymer. In an embodiment, the compositions of the present invention additionally comprise at least one inorganic salt(s) and/or filler(s).

The present invention relates to novel pharmaceutical sustained release composition of active agents preferably those having good bioavailability. In an embodiment, wherein the active agent is an antiviral, the invention relates further to a method of administering an antiviral drug composition according to the present invention to a patient infected with a virus to alleviate or at least minimize the viral infection in the patient.

The composition is formulated into a suitable dosage form and provides therapeutic concentrations of active agent(s) for extended periods of time. The novel compositions of the present invention release the active agent for a period of about 6-20 hours, preferably from about 10-16 hours. The release is primarily by diffusion followed by erosion such that the active agent leaches into the surrounding environment as long as the polymer blend containing the active agent erodes out of the formulation in a controlled manner. The polymer system used in the present invention is unique and acts to produce the desired release profile of the active agent. The compositions of the present invention are suitable preferably for water soluble drugs but sparingly water soluble and water insoluble drugs are also contemplated within the scope of the present invention. In an embodiment, the composition is a sustained release preparation wherein the drug is first granulated or coated with pH independent polymer to provide the first external barrier. Then, this blend is mixed with a sustaining system comprising a blend of anionic and cationic polymer alongwith divalent cations to provide the external barrier to drug release and to reduce the chances of dose dumping. In an embodiment, the compositions of the present invention are preferably useful for active agents for which the stomach and/or the upper part of the gastrointestinal tract are the preferred site of absorption. In another embodiment, the compositions of the present invention are formulated as gastroretentive dosage forms, wherein the said dosage form is retained for a prolonged duration in the gastrointestinal tract thus providing a sustained or controlled release of the active agent(s).

In an embodiment, the filler(s) used in the present invention is selected from but not limited to a group comprising lactose, mannitol, sorbitol, starch, microcrystalline cellulose, xylitol, fructose, sucrose, dextrose, dicalcium phosphate, calcium sulphate and the like or mixtures thereof.

In another embodiment, the pH independent polymer of the present invention is selected from but not limited to a group comprising cellulosic polymers and the like. The pH independent polymer(s) is selected from but not limited to a group comprising hydroxypropylmethyl cellulose; hydroxypropylethyl cellulose; carboxyalkylcelluloses such as carboxymethyl cellulose, carboxyethyl cellulose and the like; polyethylene glycols (PEG® 6000, PEG® 10000), copolymers of ethylene oxide with propylene oxide (Poloxamer 407, Poloxamer 188 or the like), gelatin, polyvinylpyrrolidones (PVP, Kollidon® 12 PF, Kollidon® 17 PF, Kollidon® K15, Kollidon® K30, Kollidon® K90), vinylpyrrolidones, vinyl acetates, polyvinylimidazoles, polyvinylpyridine N-oxides, copolymers of vinylpyrrolidone with long-chained alpha-olefins, copolymers of vinylpyrrolidone with vinylimidazole, poly(vinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyacrylamides, polyvinyl alcohols (PVA, Mowiol® 40-88), optionally hydrolyzed polyvinyl acetate, copolymers of ethyl acrylate with methacrylate and methacrylic acid, copolymers of maleic acid with unsaturated hydrocarbons and mixed polymerization products of the said polymers, polysaccharide gums, both natural and modified (semi-synthetic), including but not limited to xanthan gum, veegum, agar, guar gum, locust bean gum, gum arabic, okra gum, alginic acid, other alginates (e.g. sodium alginate HVCR, propyleneglycol alginate), bentonite, arabinoglactin, pectin, tragacanth, scleroglucan, dextran, amylose, amylopectin, dextrin, cyclodextrins and the like or suitable mixtures thereof.

In a preferred embodiment of the present invention, the cellulosic polymer of the present invention is selected from but not limited to a group comprising hydroxyalkylcelluloses such as hydroxypropylcellulose, hydroxymethylcellulose, hydroxyethylcellulose; alkylcelluloses such as ethyl cellulose (Aquacoat®, an aqueous dispersion of ethylcellulose available from FMC and Surelease® with different grades such as E-7-7050, E-7-7060, E-7-7100, E-7-19010, E-7-19060 an aqueous dispersion of ethylcellulose available from Colorcon), methylcellulose and the like; hydroxypropylmethyl cellulose; hydroxypropylethyl cellulose; carboxyalkylcelluloses such as carboxymethylcellulose, carboxyethylcellulose and the like; or suitable mixtures thereof. In an embodiment, the cellulosic polymer(s) used in the present invention forms a thin barrier layer of the polymer on the active agent and controls the initial burst release of the active agent.

In an embodiment of the present invention, the sustaining system comprises at least one gum. In another embodiment of the present invention, the sustaining system further comprises a methacrylic acid polymer. In another embodiment of the present invention, the sustaining system comprises an anionic gum and a cationic or a neutral methacrylic acid polymer. In yet another embodiment of the present invention, the sustaining system comprises a gum alongwith an ion exchange resin.

In a further embodiment, the gum used in the present invention is selected from but not limited to a group comprising xanthan gum, guar gum, gum arabic, carrageenan gum, karaya gum, locust bean gum, acacia gum, tragacanth gum, agar and the like or mixtures thereof.

In a further embodiment, the methacrylic acid polymer of the sustaining system is selected from but not limited to a group comprising anionic, cationic, neutral or zwitterionic polymers. In an embodiment, the polymer is selected from but not limited to a group comprising ammoniomethyacrylate copolymer such as Eudragit® EPO, Eudragit® RL or Eudragit® RS), methacrylic acid esters neutral copolymer such as Eudragit® NE30D, dimethylaminoethylmethacrylate-methacrylic acid esters copolymer, Eudragit® RLPO, Eudragit® RSPO, or mixtures thereof.

In another embodiment, the ion exchange resin is selected from but not limited to cation exchange resins such as Amberlite® IR 120B, Amberlite® IR 200C, Amberlite® IRA 68, Amberlite® IRP 64, Dowex® 50W, Dowex® MSC-1, DouLite® C-20, DouLite® C-25D and anion exchange resins such as Amberlite® IRA400, Amberlite® IRA 900, Dowex® 1, DouLite® A-101D, Duolite® AP143, Duolite® A-7, Indion® 454, Amberlite® IRA 68 and Amberlite® IRA 45, or mixtures thereof.

Other polymers that can be used in the sustaining system of the present invention are selected from but not limited to a group comprising hydrophilic polysaccharides such as alginates, chitosan, scleroglucan and semi-synthetic polysaccharides, in particular cellulose or cellulose derivatives such as methylhydroxyethylcellulose, carboxymethylcellulose and its salts such as sodium carboxymethylcellulose or calcium carboxymethylcellulose, hydroxypropylcellulose or hydroxypropylmethyl cellulose, or synthetic hydrophilic polymers such as polyvinylpyrrolidones, polymers derived from acrylic acid and methacrylic acid and salts thereof, such as polyacrylates (Carbopol®) or aminoacid polymers such as polylysines, and vinyl methyl ether/maleic anhydride copolymers or mixtures thereof.

Examples of suitable inorganic salt used in the present invention include but not limited to calcium salt, zinc salt, iron salt, magnesium salt, barium salt, strontium salt, sodium salt, potassium salt and the like or mixtures thereof. Preferably the inorganic salts are in the form of sulphates, phosphates, acetates, carbonates, oxides, hydroxides, hydrochlorides used either alone or in combination thereof.

Pharmaceutically acceptable excipients as used in the composition of the present invention are selected from a group of excipients generally used by persons skilled in the art e.g. diluents, disintegrants, binders, fillers, bulking agent, organic acid(s), colorants, stabilizers, preservatives, lubricants, glidants, chelating agents and the like. The disintegrants used in the present invention include but not limited to starch, partially pregelatinized maize starch (Starch 1500®), croscarmellose sodium, sodium starch glycollate, and the like. The lubricants used in the present invention include but not limited to talc, magnesium stearate, calcium stearate, stearic acid, hydrogenated vegetable oil and the like.

Xanthan gum is anionic which controls the release of drug by swelling mechanism. Eudragit EPO is cationic polymer, which interacts with xanthan gum and forms a gel. Calcium sulfate is water insoluble inorganic material that provides divalent cations to xanthan gum and increases the viscosity of gel, provides strength to the gel formed and inhibits the early fragmentation of the gel thereby reducing the drug release variability between individual dosage forms by maintaining the integrity of the dosage form.

The pharmaceutical compositions of the present invention may be formulated as an oral dosage form such on tablets, capsules, patches and the like. In an embodiment, the composition of the present invention is in the form of tablets. The tablets can be prepared by either direct compression, dry compression (slugging), or by granulation. The granulation technique is either aqueous or non-aqueous. The non-aqueous solvent used is selected from a group comprising ethanol; isopropyl alcohol or methylene chloride. In an embodiment, the compositions of the present invention are in the form of compressed tablets, molded tablets, products prepared by extrusion or film cast technique, and the like.

In a further embodiment, the present invention also provides process for preparation of such composition. In an embodiment, the process comprises granulation of active agent(s) or optionally a mixture of active agent(s) with a pH independent polymer(s), mixing the granules thus obtained with sustaining system and inorganic salt(s) optionally with other pharmaceutically acceptable excipients, and formulation of the mixture into a suitable dosage form.

In yet another embodiment of the present invention is provided method of using such compositions. The compositions comprising the antiviral drugs such as acyclovir, famciclovir, valacyclovir, penciclovir, ganciclovir and the like are useful in the treatment of viral infections such as HIV infections. The compositions comprising a histamine H₂-receptor antagonist such as cimetidine, ranitidine and the like are used for the treatment of ulcers, gastroesophageal reflux disease (GERD) and erosive esophagitis. The composition of the present invention comprising captopril is used to prevent the conversion of angiotensin I to angiotensin II by inhibition of ACE, a peptidyldipeptide carboxyhydrolase, and thus show beneficial effects in hypertension and heart failure. The compositions of the present invention comprising metformin are useful as oral antihyperglycemic drugs in the management of type 2 diabetes. Compositions comprising bupropion are useful as non-nicotine aid to smoking cessation. Compositions comprising tramadol are useful as opioid analgesics. Compositions comprising oxcarbazepine and levetiracetam are useful for the treatment of seizures. Compositions comprising fexofenadine are useful as histamine H₁-receptor antagonist.

In a further embodiment is provided the use of the compositions of the present invention for the preparation of medicament for the treatment of one or more diseases or disorders selected from viral infections, ulcers, gastroesophageal reflux disease (GERD), erosive esophagitis, to prevent the conversion of angiotensin I to angiotensin II by inhibition of ACE, treatment of heart failure, management of type 2 diabetes and as non-nicotine aid to smoking cessation depending on the active agent used in the composition. In an embodiment, the compositions of the present invention are useful against HUV infections.

The examples given below serve to illustrate embodiments of the present invention. However they do not intend to limit the scope of the present invention.

### EXAMPLES

### Example 1

| S. No. | Ingredients | mg/tablet |
|---|---|---|
| 1. | Famciclovir | 125.0 |
| 2. | Lactose | 5.6 |
| 3. | Aqueous ethylcellulose dispersion | 4.0 |
| | (Surelease ® E-7-19010) | |
| 4. | Xanthan gum | 9.8 |
| 5. | Calcium sulfate | 11.5 |
| 6. | Methacrylic acid polymer (Eudragit ® EPO) | 3.4 |
| 7. | Magnesium stearate | 0.9 |

### Procedure:

i) Famciclovir and Lactose are granulated with Aqueous ethylcellulose dispersion and dried.
ii) Xanthan gum and Calcium sulfate are mixed together and Methacrylic acid polymer was added thereafter and mixed well.
iii) The above mixture of step (ii) was slugged and deslugged through sieve 22 and mixed with the dried granules of step (i).
iv) The above blend of step (iii) was lubricated with Magnesium stearate and compressed into tablets.

### Example 2

| S. No. | Ingredients | mg/tablet |
|---|---|---|
| 1. | Famciclovir | 250.0 |
| 2. | Lactose | 81.0 |
| 3. | Aqueous ethylcellulose dispersion | 44.0 |
| | (Surelease ® E-7-19060) | |
| 4. | Guar gum | 50.0 |
| 5. | Calcium sulfate | 20.0 |
| 6. | Methacrylic acid polymer (Eudragit ® RSPO) | 40.0 |
| 7. | Zinc stearate | 10.0 |

### Procedure:

i) Famciclovir and Lactose are granulated with Aqueous ethylcellulose dispersion and dried.
ii) Guar gum and Calcium sulfate are mixed together and Methacrylic acid polymer was added thereafter and mixed well.
iii) The above mixture of step (ii) was slugged and deslugged through sieve 22 and mixed with the dried granules of step (i).
iv) The above blend of step (iii) was lubricated with Zinc stearate and compressed into tablets.

### Example 3

| S. No. | Ingredients | mg/tablet |
|---|---|---|
| 1. | Acyclovir | 800.0 |
| 2. | Dextrose | 51.0 |
| 3. | Aqueous hydroxypropylmethyl cellulose dispersion | 44.0 |
| 4. | Guar gum | 80.0 |
| 5. | Magnesium sulfate | 120.0 |
| 6. | Methacrylic acid polymer (Eudragit ® RLPO) | 40.0 |
| 7. | Magnesium stearate | 10.0 |

### Procedure:

i) Acyclovir and Dextrose are granulated with Aqueous hydroxypropylmethyl cellulose dispersion and dried.
ii) Guar gum and Magnesium sulfate are mixed together and Methacrylic acid polymer was added thereafter and mixed well.
iii) The above mixture of step (ii) was mixed with granules of step (i). The blend was slugged and deslugged through sieve 22.
iv) The above granules of step (iii) was lubricated with Magnesium stearate and compressed into tablets.

### Example 4

| S. No. | Ingredients | mg/capsule |
|---|---|---|
| 1. | Ganciclovir | 500.0 |
| 2. | Lactose | 55.0 |
| 3. | Polyvinylpyrrolidone (Kollidon ® K15) | 40.0 |
| 4. | Xanthan gum | 80.0 |
| 5. | Potassium phosphate | 20.0 |
| 6. | Ion exchange resin (Amberlite ® IR 120B) | 40.0 |
| 7. | Hydroxypropylmethyl cellulose | 30.0 |
| 8. | Partially pregelatinized maize starch | 170.0 |
| | (Starch 1500 ®) | |
| 9. | Zinc stearate | 10.0 |

### Procedure:

i) Ganciclovir and Lactose are granulated with Polyvinylpyrrolidone and dried.
ii) Xanthan gum and Potassium phosphate are mixed together and Ion exchange resin was added thereafter and mixed well followed by addition and mixing of Hydroxypropylmethyl cellulose, Partially pregelatinized maize starch and Zinc stearate.
iii) The above mixture of step (ii) was mixed with granules of step (i).
iv) The above blend of step (iii) was lubricated with Zinc stearate and filled into hard gelatin capsules.

### Example 5

| S. No. | Ingredients | mg/capsule |
|---|---|---|
| 1. | Ganciclovir | 500.0 |
| 2. | Lactose | 75.0 |
| 3. | Polyvinylpyrrolidone (Kollidon ® K15) | 40.0 |
| 4. | Xanthan gum | 80.0 |
| 5. | Methacrylic acid polymer (Eudragit ® RS) | 40.0 |
| 6. | Hydroxypropylmethyl cellulose | 30.0 |
| 7. | Partially pregelatinized maize starch | 170.0 |
| | (Starch 1500 ®) | |
| 8. | Zinc stearate | 10.0 |

### Procedure:

i) Ganciclovir and Lactose are granulated with Polyvinylpyrrolidone and dried.
ii) Methacrylic acid polymer was added to Xanthan gum and mixed well followed by addition and mixing of Hydroxypropylmethyl cellulose, Partially pregelatinized maize starch and Zinc stearate.
iii) The above mixture of step (ii) was mixed with granules of step (i).
iv) The above blend of step (iii) was lubricated with Zinc stearate and filled into hard gelatin capsules.

### Example 6

| S. No. | Ingredients | mg/tablet |
|---|---|---|
| 1. | Valacyclovir hydrochloride | 556.0 |
| | (Equivalent to 500.0 mg Valacyclovir) | |
| 2. | Sucrose | 96.0 |
| 3. | Sodium carboxymethylcellulose | 26.0 |
| 4. | Locust bean gum | 50.0 |
| 5. | Calcium carbonate | 20.0 |
| 6. | Methacrylic acid polymer (Eudragit ® EPO) | 70.0 |
| 7. | Hydroxypropylmethyl cellulose (HPMC E5) | 70.0 |
| 8. | Stearic acid | 10.0 |

### Procedure:

i) Valacyclovir hydrochloride and Sucrose are mixed together and granulated with Sodium carboxymethylcellulose and dried in tray drier. Other ingredients of the formulation were sifted through sieve #40.
ii) Locust bean gum and Calcium carbonate were mixed together followed by addition and mixing of Methacrylic acid polymer and Hydroxypropylmethyl cellulose.
iii) The mixture of step (ii) was slugged and deslugged and granules were passed through sieve #30.
iv) The above granules of step (iii) were then mixed with dried granules of (i).
v) After mixing, blend of step (iv) was lubricated with Stearic acid and then compressed into tablets.

### Example 7

| S. No. | Ingredients | mg/tablet |
|---|---|---|
| 1. | Valacyclovir hydrochloride | 556.0 |
| | (Equivalent to 500.0 mg Valacyclovir) | |
| 2. | Sodium carboxymethylcellulose | 86.0 |
| 3. | Locust bean gum | 50.0 |
| 4. | Calcium carbonate | 116.0 |
| 5. | Hydroxypropylmethyl cellulose (HPMC E5) | 70.0 |
| 6. | Stearic acid | 10.0 |

### Procedure:

i) Valacyclovir hydrochloride is granulated with Sodium carboxymethylcellulose and dried in tray drier. Other ingredients of the formulation were sifted through sieve #40.
ii) Locust bean gum and Calcium carbonate were mixed together followed by addition and mixing of Hydroxypropylmethyl cellulose.
iii) The mixture of step (ii) was slugged and deslugged and granules were passed through sieve #30.
iv) The above granules of step (iii) were then mixed with dried granules of (i).
v) After mixing, blend of step (iv) was lubricated with Stearic acid and then compressed into tablets.

### Example 8

| S. No. | Ingredients | mg/tablet |
|---|---|---|
| 1. | Ranitidine hydrochloride | 336.0 |
| | (Equivalent to 300.0 mg Ranitidine) | |
| 2. | Mannitol | 80.0 |
| 3. | Polyvinyl alcohol (Mowiol ® 40) | 42.0 |
| 4. | Guar gum | 50.0 |
| 5. | Magnesium oxide | 20.0 |
| 6. | Methacrylic acid polymer (Eudragit ® EPO) | 40.0 |
| 7. | Hydroxypropylethyl cellulose | 30.0 |
| 8. | Magnesium stearate | 10.0 |
| 9. | Sodium starch glycollate | 70.0 |

### Procedure:

i) Ranitidine hydrochloride and Mannitol are mixed together, granulated with Polyvinyl alcohol and then dried in tray drier. Other ingredients of the formulation were sifted through sieve #40.
ii) Guar gum and Magnesium oxide were mixed together followed by addition and mixing of Methacrylic acid polymer and Hydroxypropylethyl cellulose.
iii) The mixture of step (ii) was slugged and deslugged and granules were passed through sieve #30.
iv) The above granules of step (iii) were then mixed with dried granules of step (i).
v) After mixing, blend of step (iv) was mixed with Magnesium stearate and Sodium starch glycollate and then compressed into tablet.

### Example 9

| S. No. | Ingredients | mg/tablet |
|---|---|---|
| 1. | Fexofenadine hydrochloride | 240.0 |
| 2. | Dextrose | 78.0 |
| 3. | Aqueous ethylcellulose dispersion | 44.0 |
| | (Surelease ® E-7-7050) | |
| 4. | Karaya gum | 50.0 |
| 5. | Calcium sulfate | 20.0 |
| 6. | Methacrylic acid polymer (Eudragit ® RLPO) | 70.0 |
| 7. | Zinc stearate | 10.0 |
| 8. | Croscarmellose sodium | 80.0 |

### Procedure:

i) Fexofenadine hydrochloride and Dextrose are mixed together, granulated with Aqueous ethylcellulose dispersion and then dried in tray drier. Other ingredients of the formulation were sifted through sieve #40.
ii) Karaya gum and Calcium sulfate were mixed together followed by addition and mixing of Methacrylic acid polymer and Croscarmellose sodium.
iii) The mixture was slugged and deslugged and granules were passed through sieve #30.
iv) The above granules of step (iii) were then mixed with dried granules of step (i).
v) After mixing, blend of step (iv) was lubricated with Zinc stearate and then compressed into tablet.

### Example 10

| S. No. | Ingredients | mg/capsule |
|---|---|---|
| 1. | Captopril | 100.0 |
| 2. | Mannitol | 83.0 |
| 3. | Hydroxypropyl cellulose (Klucel ®) | 39.0 |
| 4. | Carrageenan gum | 50.0 |
| 5. | Calcium chloride | 20.0 |
| 6. | Methacrylic acid polymer (Eudragit ® RSPO) | 40.0 |
| 7. | Calcium stearate | 10.0 |
| 8. | Partially pregelatinized maize starch | 70.0 |
| | (Starch 1500 ®) | |

### Procedure:

i) Captopril and Mannitol are mixed together, granulated with Hydroxypropyl cellulose and then dried in tray drier. Other ingredients of the formulation were sifted through sieve #40.
ii) Carrageenan gum and Calcium chloride were mixed together followed by addition and mixing of Methacrylic acid polymer and Partially pregelatinized maize starch.
iii) The mixture of step (ii) was slugged and deslugged and granules were passed through sieve #30.
iv) The above granules of step (iii) were then mixed with dried granules of step (i).
v) After mixing, blend of step (iv) was lubricated with Calcium stearate and then filled into hard gelatin capsules.

### Example 11

| S. No. | Ingredients | mg/tablet |
|---|---|---|
| 1. | Bupropion hydrochloride | 150.0 |
| 2. | Lactose | 78.0 |
| 3. | Aqueous ethylcellulose dispersion | 44.0 |
| | (Surelease ® E-7-19010) | |
| 4. | Acacia gum | 50.0 |
| 5. | Potassium sulfate | 20.0 |
| 6. | Methacrylic acid polymer (Eudragit ® RS) | 40.0 |
| 7. | Magnesium stearate | 10.0 |

### Procedure:

i) Bupropion hydrochloride and Lactose are mixed together, granulated with Aqueous ethylcellulose dispersion and then dried in tray drier. Other ingredients of the formulation were sifted through sieve #40.
ii) Acacia gum and Potassium sulfate were mixed together followed by addition and mixing of Methacrylic acid polymer.
iii) The mixture of step (ii) was slugged and deslugged and granules were passed through sieve #30.
iv) The above granules of step (iii) were then mixed with dried granules of step (i).
v) After mixing, blend of step (iv) was lubricated with Magnesium stearate and then compressed into tablet.

### Example 12

| S. No. | Ingredients | mg/tablet |
|---|---|---|
| 1. | Metformin hydrochloride | 150.0 |
| 2. | Mannitol | 78.0 |
| 3. | Polyethylene glycol (PEG ® 6000) | 44.0 |
| 4. | Karaya gum | 50.0 |
| 5. | Potassium sulfate | 20.0 |
| 6. | Methacrylic acid polymer (Eudragit ® RSPO) | 40.0 |
| 7. | Zinc stearate | 10.0 |
| 8. | Sodium starch glycollate | 70.0 |

### Procedure:

i) Metformin hydrochloride and Mannitol are mixed together, granulated with Polyethylene glycol and then dried in tray drier. Other ingredients of the formulation were sifted through sieve #40.
ii) Karaya gum and Potassium sulfate were mixed together followed by addition and mixing of Methacrylic acid polymer and Sodium starch glycollate.
iii) The mixture of step (ii) was slugged and deslugged and granules were passed through sieve #30.
iv) The above granules of step (iii) were then mixed with dried granules of step (i).
v) After mixing, blend of step (iv) was lubricated with Zinc stearate and then compressed into tablet.

### Example 13

| S. No. | Ingredients | mg/tablet |
|---|---|---|
| 1. | Oxcarbazepine | 60.0 |
| 2. | Lactose | 16.0 |
| 3. | Aqueous ethylcellulose dispersion | 3.0 |
| | (Surelease ® E-7-19010) | |
| 4. | Locust bean gum | 5.0 |
| 5. | Calcium sulfate | 10.0 |
| 6. | Methacrylic acid polymer (Eudragit ® RL) | 50.0 |
| 7. | Calcium stearate | 1.0 |

### Procedure:

i) Oxcarbazepine and Lactose are mixed together, granulated with Aqueous ethylcellulose dispersion and then dried in tray drier. Other ingredients of the formulation were sifted through sieve #40.
ii) Locust bean gum and Calcium sulfate were mixed together followed by addition and mixing of Methacrylic acid polymer.
iii) The mixture of step (ii) was slugged and deslugged and granules were passed through sieve #30.
iv) The above granules of step (iii) were then mixed with dried granules of step (i).
v) After mixing, blend of step (iv) was lubricated with Calcium stearate and then compressed into tablet.

### Example 14

| S. No. | Ingredients | mg/tablet |
|---|---|---|
| 1. | Leveteracetam | 66.98 |
| 2. | Mannitol | 2.01 |
| 3. | Aqueous ethylcellulose dispersion | 5.00 |
| | (Surelease ® E-7-7050) | |
| 4. | Guar gum | 5.00 |
| 5. | Calcium sulfate | 15.00 |
| 6. | Methacrylic acid polymer (Eudragit ® RLPO) | 5.00 |
| 7. | Magnesium stearate | 1.00 |

### Procedure:

i) Leveteracetam and Mannitol are mixed together, granulated with Aqueous ethylcellulose dispersion and then dried in tray drier. Other ingredients of the formulation were sifted through sieve #40.
ii) Guar gum and Calcium sulfate were mixed together followed by addition and mixing of Methacrylic acid polymer.
iii) The mixture of step (ii) was slugged and deslugged and granules were passed through sieve #30.
iv) The above granules of step (iii) were then mixed with dried granules of step (i).
v) After mixing, blend of step (iv) was lubricated with Magnesium stearate and then compressed.

### Example 15

| S. No. | Ingredients | mg/tablet |
|---|---|---|
| 1. | Tramadol HCl | 20.0 |
| 2. | Lactose | 52.0 |
| 3. | Hydroxypropylmethyl cellulose | 4.0 |
| | (Methocel ® K15M) | |
| 4. | Tragacanth gum | 10.0 |
| 5. | Potassium sulfate | 10.0 |
| 6. | Methacrylic acid polymer (Eudragit ® NE30D) | 3.0 |
| 7. | Zinc stearate | 1.0 |

### Procedure:

i) Tramadol HCl and Lactose are mixed together, granulated with Hydroxypropylmethyl cellulose and then dried in tray drier. Other ingredients of the formulation were sifted through sieve #40.
ii) Tragacanth gum and Potassium sulfate were mixed together followed by addition and mixing of Methacrylic acid polymer.
iii) The mixture of step (ii) was slugged and deslugged and granules were passed through sieve #30.
iv) The above granules of step (iii) were then mixed with dried granules of step (i).
v) After mixing, blend of step (iv) was lubricated with Zinc stearate and then compressed.

### Example 16

| S. No. | Ingredients | mg/tablet |
|---|---|---|
| 1. | Metformin HCl | 66.00 |
| 2. | Dextrose | 6.00 |
| 3. | Aqueous ethylcellulose dispersion | 3.80 |
| | (Surelease ® E-7-7050) | |
| 4. | Locust bean gum | 10.60 |
| 5. | Calcium sulfate | 10.56 |
| 6. | Methacrylic acid polymer (Eudragit ® EPO) | 3.00 |
| 7. | Calcium stearate | 1.00 |

### Procedure:

i) Metformin HCl and Dextrose are mixed together, granulated with Aqueous ethylcellulose dispersion and then dried in tray drier. Other ingredients of the formulation were sifted through sieve #40.
ii) Locust bean gum and Calcium sulfate were mixed together followed by addition and mixing of Methacrylic acid polymer.
iii) The mixture of step (ii) was slugged and deslugged and granules were passed through sieve #30.
iv) The above granules of step (iii) were then mixed with dried granules of step (i).
v) After mixing, blend of step (iv) was lubricated with Calcium stearate and then compressed.

### Example 17

| S. No. | Ingredients | mg/tablet |
|---|---|---|
| 1. | Ranitidine hydrochloride | 336.0 |
| | (Equivalent to 300.0 mg Ranitidine) | |
| 2. | Mannitol | 80.0 |
| 3. | Polyvinyl alcohol (Mowiol ® 40) | 42.0 |
| 4. | Guar gum | 50.0 |
| 5. | Magnesium oxide | 20.0 |
| 6. | Ion exchange resin (Amberlite ® IR 200C) | 40.0 |
| 7. | Hydroxypropylethyl cellulose | 30.0 |
| 8. | Magnesium stearate | 10.0 |
| 9. | Sodium starch glycollate | 70.0 |

### Procedure:

i) Ranitidine hydrochloride and Mannitol are mixed together, granulated with Polyvinyl alcohol and then dried in tray drier. Other ingredients of the formulation were sifted through sieve #40.
ii) Guar gum and Magnesium oxide were mixed together followed by addition and mixing of Ion exchange resin and Hydroxypropylethyl cellulose.
iii) The mixture of step (ii) was slugged and deslugged and granules were passed through sieve #30.
iv) The above granules of step (iii) were then mixed with dried granules of step (i).
v) After mixing, blend of step (iv) was mixed with Magnesium stearate and Sodium starch glycollate and then compressed into tablet.

1-21. (canceled)

22. A pharmaceutical sustained release composition comprising at least one active agent(s), or a tautomeric form, analog, isomer, polymorph, solvate, derivative, or salt thereof; at least one pH independent polymer(s); a sustaining system consisting of a gum along with at least one methacrylic acid polymer(s) or an ion exchange resin; optionally with other pharmaceutically acceptable excipient(s); wherein said pH independent polymer(s) forms a thin barrier on the active agent and controls the initial burst release of the active agent, and wherein the components of said sustaining system interact to form a gel, and wherein said inorganic salt(s) increases the viscosity and provides strength to the gel thus formed.

23. The composition according to claim 22, wherein the active agent is selected from a group comprising acyclovir, famciclovir, valacyclovir, penciclovir, ganciclovir, ritonavir, lopinavir, saquinavir, cimetidine, ranitidine, captopril, metformin, bupropion, fexofenadine, oxcarbazepine, levetiracetam, tramadol, or a tautomeric form, analog, isomer, polymorph, solvate, derivative, or salt thereof, used either alone or in combination thereof.

24. The composition according to claim 22, wherein the pH independent polymer is selected from a group comprising cellulosic polymers, polyethylene glycols, copolymers of ethylene oxide with propylene oxide, gelatin, polyvinylpyrrolidones, vinylpyrrolidones, vinyl acetates, polyvinylimidazoles, polyvinylpyridine N-oxides, copolymers of vinylpyrrolidone with long-chained alpha-olefins, copolymers of vinylpyrrolidone with vinylimidazole, poly(vinylpyrrolidone/dimethylaminoethyl methacrylates), copolymers of vinylpyrrolidone/dimethylaminopropyl methacrylamides, copolymers of vinylpyrrolidone/dimethylaminopropyl acrylamides, quaternised copolymers of vinylpyrrolidones and dimethylaminoethyl methacrylates, terpolymers of vinylcaprolactam/vinylpyrrolidone/dimethylaminoethyl methacrylates, copolymers of vinylpyrrolidone and methacrylamidopropyl-trimethylammonium chloride, terpolymers of caprolactam/vinylpyrrolidone/dimethylaminoethyl methacrylates, copolymers of styrene and acrylic acid, polycarboxylic acids, polyacrylamides, polyvinyl alcohols, optionally hydrolyzed polyvinyl acetate, copolymers of ethyl acrylate with methacrylate and methacrylic acid, copolymers of maleic acid with unsaturated hydrocarbons and mixed polymerization products of the polymers, and polysaccharide gums, or a mixture thereof.

25. The composition according to claim 24, wherein the cellulosic polymer is selected from a group comprising hydroxypropylmethyl cellulose, and hydroxypropylethyl cellulose, or a mixture thereof.

26. The composition according to claim 24, wherein the cellulosic polymer is selected from a group comprising hydroxyalkylcelluloses, alkylcelluloses, and carboxyalkylcelluloses, or a mixture thereof.

27. The composition according to claim 22, wherein the gum is selected from a group comprising xanthan gum, veegum, agar, guar gum, locust bean gum, gum arabic, okra gum, alginic acid or a derivative thereof, arabinogalactan, pectin, tragacanth, scleroglucan, dextran, amylose, and amylopectin, or a mixture thereof.

28. The composition according to claim 24, wherein the gum is selected from a group comprising xanthan gum, veegum, agar, guar gum, locust bean gum, gum arabic, okra gum, alginic acid or a derivative thereof, arabinogalactan, pectin, tragacanth, scleroglucan, dextran, amylose, and amylopectin, or a mixture thereof.

29. The composition according to claim 22, further comprising at least one inorganic salt, one filler or both.

30. The composition according to claim 29, wherein the inorganic salt is selected from a group comprising calcium salt, zinc salt, iron salt, magnesium salt, barium salt, strontium salt, sodium salt, and potassium salt, or a mixture thereof.

31. The composition according to claim 29, wherein the filler is selected from a group comprising lactose, mannitol, sorbitol, starch, microcrystalline cellulose, xylitol, fructose, sucrose, dextrose, dicalcium phosphate, and calcium sulphate, or a mixture thereof.

32. The composition according to claim 22, wherein the methacrylic acid polymer is selected from a group comprising ammoniomethyacrylate copolymer, methacrylic acid esters neutral copolymer, and dimethylaminoethylmethacrylate-methacrylic acid esters copolymer, or a mixture thereof.

33. The composition according to claim 22, wherein the ion exchange resin is a cation exchange resin or an anion exchange resin.

34. The composition according to claim 22, wherein the pharmaceutically acceptable excipients are selected from a group comprising diluents, disintegrants, binders, fillers, bulking agents, organic acid(s), colorants, stabilizers, preservatives, lubricants, glidants, and chelating agents, or a mixture thereof.

35. A process for preparing a sustained release composition according to claim 22, comprising at least one active agent, a tautomeric form, analog, isomer, polymorph, solvate, derivative, or salt thereof; at least one pH independent polymer; a sustaining system consisting of a gum alongwith at least one methacrylic acid polymer or an ion exchange resin; optionally with other pharmaceutically acceptable excipient(s); which comprises the steps of:
i) granulation the active agent or a mixture of active agents with one or more pH independent polymer;
ii) mixing the granules obtained in step i) thus with a sustaining system, optionally with one or more inorganic salt, other pharmaceutically acceptable excipients, or both; and
iii) formulating the mixture of step ii) into a dosage form.

36. A method of using the pharmaceutical composition according to claim 1, which comprises administering to a patient in need thereof an effective amount of the composition.
